(19) United States
(12) Patent Application Publication
(10) Pub. No.: US 2017/0355955 A1
(43) Pub. Date: Dec. 14, 2017

(54) DEVICE AND METHOD FOR SELECTING EUKARYOTIC CELLS IN A TRANSPORTATION CHANNEL BY ALTERING THE EUKARYOTIC CELLS BY MEANS OF ELECTROMAGNETIC RADIATION

(71) Applicant: GENES DIFFUSION, DOUAI (FR)

(72) Inventors: Luc LIEGEOIS, Raimbeaucourt (FR); Emmanuel COURTADE, Lille-Hellemmes (FR); Herve DAMART, Wambrechies (FR); Jean PESEZ, Lambersart (FR); Quentin THOMMEN, Lille-Hellemmes (FR); Anthony TREIZEBRE, Coutiches (FR)

(21) Appl. No.: 15/501,276

(22) PCT Filed: Aug. 4, 2015
(86) PCT No.: PCT/FR2015/052154

§ 371 Date: Jun. 30, 2017

(30) Foreign Application Priority Data
Aug. 11, 2014 (FR) 1457734

(51) Int. Cl. C12N 5/071; G01N 15/14; C12M 1/42

(52) U.S. Cl. CPC C12N 5/0612; G01N 15/1459; G01N 15/1484; G01N 2015/149; G01N 15/14; C12M 1/42

(57) ABSTRACT

The device can be used for the in vitro selection of eukaryotic cells, and in particular sperm cells (SP). It comprises a transportation channel (100), preferably a DE TR transportation channel (100), in which a solution containing said eukaryotic cells can circulate (SP), a first through passage (104) opening into said transportation channel (100), a source of electromagnetic radiation (60), which is coupled to a first end of a first optical fibre (61), the other emission end (61b) of the first optical fibre being inserted into said first through passage (104), without protruding into the transportation channel (100). The device further comprises electronic control means (7), which make it possible to automatically control said source of electromagnetic radiation (60), so as to selectively alter the eukaryotic cells (SP) circulating in the transportation channel (100), by means of the electromagnetic alteration radiation (R) emitted by the source of electromagnetic radiation (60).

### TECHNICAL FIELD

The present invention relates to the field of the in vitro selection of eukaryotic cells, and in particular, but not exclusively, sperm cells, in particular animal sperm cells, using electromagnetic radiation.

### PRIOR ART

A sperm cell is a eukaryotic cell of the haploid type, which generally contains only one copy of each chromosome, for example an X or Y chromosome, whose motility is ensured by a flagellum.

In the field of animal reproduction, different sex selection solutions have been developed to date, making it possible to select and sort sperm cells automatically based on their X or Y chromosome, and which are based on flux cytometry.

In general, all of these technical sex selection solutions by flux cytometry are based on the fact that X sperm cells contain about 4% more DNA than Y sperm cells. Thus, in these technical sex selection solutions:
the sperm cells are marked with a fluorescent DNA intercalary, generally called fluorochrome; the X sperm cells, which are presumed to contain a larger quantity of DNA, absorb a larger quantity of fluorochrome than the Y sperm cells;
a solution containing the marked sperm cells is injected into a transportation channel, and if applicable subjected to hydrodynamic focusing so as to align them and cause them to circulate in this transportation channel;
the marked sperm cells are excited with appropriate radiation, so that they emit light radiation by fluorescence;
the intensity of the light radiation emitted by fluorescence (fluorescence intensity) is detected, and
each X or Y sperm cell is selected automatically based on this detected fluorescence intensity.

A first known sorting method (aforementioned step (e)) consists of subjecting the solution containing the marked sperm cells to vibrations using a piezoelectric transducer, so as to form microdroplets, which, to the fullest extent possible, each contain only one sperm cell, then electrically charging each droplet based on the detected fluorescent intensity, and lastly successively passing the electrically charged microdroplets in an electric field making it possible to separate the charged droplets containing an X sperm cell from the charged droplets containing a Y sperm cell.

This sorting method is for example described in the following international patent applications: WO2004/104178, WO2004/017041, WO2009/014643, WO2009/151624.

This sorting method by forming microdroplets has several drawbacks. The microdroplet formation step is delicate, and it is difficult to ensure that a microdroplet contains only one sperm cell. Microdroplet formation is a limiting factor for the sorting rhythm. In this method, the sperm cells are subjected to a substantial mechanical stress, which may damage them irreversibly and uncontrollably.

In international patent application WO2010/001254, another solution was proposed in which the sperm cell selection is done using electromagnetic radiation of the laser beam type, chosen so as to selectively alter, based on the detected fluorescent intensity, the marked sperm cells transported in a single-file line in a transportation channel. This solution advantageously makes it possible to avoid the drawbacks inherent to microdroplet formation. Nevertheless, this selection solution using a laser has other drawbacks. The energy of the electromagnetic radiation used to selectively alter the sperm cells is preponderantly absorbed by the sperm cell transportation fluid and by the wall of the transportation channel traversed by the electromagnetic radiation, which normally leads to having to use high-power lasers to alter the sperm cells. Yet using high-power lasers is a limiting factor for the selection rhythm, since the switching times of a laser increase with the power of the laser. Furthermore, the alignment of the laser and its focusing optics relative to the transportation channel are delicate and highly sensitive, and the slightest optical misalignment may significantly harm the effectiveness of the laser. To the applicant's knowledge, this technical solution is not commercially used at this time.

Sorting solutions have also been proposed using electromagnetic radiation making it possible to selectively deviate, based on the detected fluorescent intensity, the trajectories of the marked and aligned sperm cells in a transportation channel, which makes it possible to orient the sperm cells automatically based on their X or Y chromosome, without altering the sperm cells and without having to form microdroplets. This type of solution requires implementing high-power electromagnetic radiation to selectively modify the trajectory of the sperm cells, and can be implemented only with a very low transportation speed of the sperm cells and with a very slow sorting rhythm. To the applicant's knowledge, this technical solution is not commercially used at this time, and the technical feasibility of this type of solution remains to be demonstrated.

The aforementioned technical solutions may also be used for the in vitro selection of any other known type of eukaryotic cells.

At this time, there is a need to find a technical solution for selecting eukaryotic cells, and in particular mammal sperm cells, in particular animal sperm cells, that is effective, and that offsets the aforementioned drawbacks of the prior art.

### Aims of the Invention

The present invention aims to propose a new technical solution for selecting eukaryotic cells that in particular offsets the aforementioned drawbacks of the prior art.

### BRIEF DESCRIPTION OF THE INVENTION

The first aim of the invention is thus a device for selecting eukaryotic cells, and in particular sperm cells, said device including a transportation channel, in which a solution containing said eukaryotic cells can circulate, a first through passage opening into said transportation channel, a source of electromagnetic radiation, which is coupled to a first end of a first optical fiber, the other emission end of the first optical fiber being inserted into said first through passage, without protruding into the transportation channel. The device further includes electronic control means, which make it possible to control said source of electromagnetic radiation automatically, so as to selectively alter the eukaryotic cells circulating in the transportation channel, by means of the electromagnetic alteration radiation emitted by the source of electromagnetic radiation.

The term “ater” or “alteration” in the present text means that the eukaryotic cell is modified directly or indirectly by the electromagnetic alteration radiation, such that its viability or motility is deteriorated enough for the eukaryotic cell to no longer be viable or fertile, independently of the physical and/or biological and/or chemical phenomenon causing this deterioration. In the general context of the invention, this alteration by the electromagnetic radiation can for example result from a lesion or photochemical alteration of the eukaryotic cell and/or an effective thermal stress experienced by the eukaryotic cell.

Preferably, the transportation channel is a microfluidic channel whereof at least one dimension in cross-section (section perpendicular to the movement direction of the sperm cells in the channel) is smaller than 1 mm, and more particularly smaller than 100 μm. [0016]Owing to the combination of a transportation channel, preferably microfluidic, and an integrated optical fiber, the electromagnetic alteration radiation can be delivered in the transportation channel as close as possible to the eukaryotic cell, which advantageously makes it possible to improve the interaction of the electromagnetic radiation with the eukaryotic cells, and as a result advantageously makes it possible to implement a lower-power source of electromagnetic radiation, which may be switched or modulated more quickly. Furthermore, in the invention, the emission end of the optical fiber being inserted into said through passage, without protruding in the transportation channel, the flow of eukaryotic cells is not disrupted, which makes it possible to maintain a precise localization of the eukaryotic cells in the transportation channel. Lastly, the emission end of the optical fiber being inserted in said through passage, the device can be manipulated without any risk of optical misalignment, and is thus robust.

The device according to the invention can be used for the in vitro selection of eukaryotic cells, and in particular sperm cells, and can particularly be used in the field of animal reproduction to select in vitro any type of animal sperm cell, and non-limitingly and non-exhaustively, bovine, porcine, ovine, equine, caprine, rabbit, bird sperm cells. The invention is not limited to the selection of sperm cells of type X or type Y, but can be used to select any other type of sperm cells.

More particularly, the device according to the invention may include the following additional and optional features, considered alone or in combination:
the transportation channel is a microfluidic channel whereof at least one dimension in cross-section is smaller than 1 mm, and more particularly smaller than 100 μm
the device includes optical focusing means, which are fixed or integrated to the emission end of the first optical fiber, and which make it possible to focus, in said transportation channel, the electromagnetic radiation emitted at the outlet of the first optical fiber.
said optical focusing means are formed by the emission end of the first optical fiber, which is profiled so as to focus, in said transportation channel, the electromechanical radiation (R) emitted at the outlet of the optical fiber.
the emission end of the first optical fiber has a conical shape.
the emission end of the first optical fiber (61) is of the “wedge” type.
the emission end of the first optical fiber is flush with the transportation channel without protruding in the transportation channel.
the distal emission part of the first optical fiber is inserted into said first through passage, abutting against a shoulder.
the transportation channel has a rectangular cross-section.
the width of the rectangular transportation channel is less than 1 mm, and preferably less than 100 μm.
the first through passage is made through one of the longitudinal walls with a larger dimension of the transportation channel with a rectangular section.
the device includes a second source of electromagnetic radiation able to emit electromagnetic excitation radiation, which makes it possible to excite the emission by fluorescence of the eukaryotic cells circulating in said transportation channel (100), and at least one photodetector making it possible to detect the fluorescence emitted by said eukaryotic cells.
the electronic control means are able to process an electrical detection signal delivered by said photodetector, and to control the first source of electromagnetic radiation, for the selective alteration of the eukaryotic cells, based on this detection signal.
the device includes a through passage for the fluorescence excitation, which opens into said transportation channel, upstream of the first through passage; the second source of electromagnetic radiation is coupled to a first end of a fluorescence excitation optical fiber, the other emission end of this fluorescence excitation optical fiber being inserted into said through passage for the fluorescence excitation, without protruding in the transportation channel.
the distal emission part of the fluorescence excitation optical fiber is inserted into said through passage for the fluorescence excitation, abutting against a shoulder.
the device includes a through passage for the fluorescence detection, which opens into said transportation channel upstream of the first through passage; one end of an optical fluorescence detection fiber is inserted into this through passage, without protruding in the transportation channel, and the other emission end of said optical fluorescence detection fiber being associated with the photodetector.
the optical fluorescence detection fiber is a large core optical fiber.
a distal part of the optical fluorescence detection fiber is inserted into said through passage for the fluorescence detection, abutting against a shoulder.
the transportation channel being defined at least by a bottom wall and by two longitudinal walls opposite one another that are transverse, and preferably perpendicular, to the bottom wall, the through passage for the fluorescence excitation is done through the bottom wall of the transportation channel, and the through passage for the fluorescence detection is made through one of the longitudinal walls of the transportation channel.
the transportation channel being defined at least by a bottom wall and two longitudinal walls opposite one another that are transverse, and preferably perpendicular, to the bottom wall, the through passage for the fluorescence detection is made through the bottom wall of the transportation channel, and the through passage for the fluorescence excitation is made through one of the longitudinal walls of the transportation channel.
the transportation channel being defined at least by a bottom wall and by two longitudinal walls opposite one another that are transverse, and preferably perpendicular, to the bottom wall, the through passage for the fluorescence excitation is done through one of the longitudinal walls of the transportation channel, and the through passage for the fluorescence detection is done through the other longitudinal wall of the transportation channel.
the distance between the outlet in the transportation channel of the through passage for the fluorescence excitation and the opposite wall of the transportation channel is less than 1 mm, preferably less than 100 μm.
the distance between the outlet in the transportation channel of the through passage for the fluorescence detection and the opposite wall of the transportation channel is smaller than 1 mm, preferably smaller than 100 μm.
the distance between the outlet in the transportation channel of the first through passage and the opposite wall of the transportation channel is less than 1 mm, preferably less than 100 μm, and still more preferably less than 50 μm.
the device includes means making it possible to inject, in said transportation channel, a solution containing eukaryotic cells, and preferably eukaryotic cells marked using at least one fluorochrome.
the device includes hydrodynamic focusing means making it possible to inject a liquid into the transportation channel so as to drive the eukaryotic cells in the transportation channel, positioning them substantially in a hydrodynamic focusing plane or substantially along a hydrodynamic focusing axis and spacing them apart one behind one another.
said optical focusing means make it possible to focus the electromagnetic alteration radiation substantially in said hydrodynamic focusing plane of the eukaryotic cells or substantially on the hydrodynamic focusing axis of the eukaryotic cells.
the transportation channel is defined in part by a slot etched in one of the faces of a hard substrate, and more particularly in a silicon substrate.

the first through passage, and if applicable the through passage for the fluorescence excitation, and/or the through passage for the fluorescence detection, are each defined in part by a slot edge in the same face of the substrate as the transportation channel.
the mean power of the source of electromagnetic alteration radiation of the eukaryotic cells is less than 10 W, and preferably less than 1 W.

The invention also relates to a method for the in vitro selection of eukaryotic cells able to have different types making it possible to inventory them in at least two different categories. More particularly, but not exclusively, the eukaryotic cells may for example be differentiated owing to the DNA of their cores. To carry out this method, the aforementioned selection device is used, and a solution containing the eukaryotic cells to be selected is injected into the transportation channel of the selection device; said eukaryotic cells are circulated in the transportation channel one after the other; the type of each eukaryotic cell circulating in the transportation channel is detected automatically; and the eukaryotic cells that have been detected as being of the same predefined type are irradiated selectively, with the electromagnetic alteration radiation, so as to alter them enough to make them nonviable, the other eukaryotic cells not being altered using the electromagnetic alteration radiation.

Before carrying out the selection method, the eukaryotic cells may have undergone a freezing or cooling treatment for conservation. Additionally, the eukaryotic cells may or may not have been purified.
More particularly, the method according to the invention may include the additional and optional features below, considered alone or in combination:
the eukaryotic cells are hydrodynamically focused in the transportation channel so as to align them behind one another substantially in a hydrodynamic focusing plane or substantially along a hydrodynamic focusing axis, and the distance between the outlet into the transportation channel of the first through passage and the hydrodynamic focusing plane or a hydrodynamic focusing axis of the eukaryotic cells in the transportation channel is less than 1 mm, more preferably less than 100 μm, and still more preferably less than 50 μm.
the eukaryotic cells are hydrodynamically focused in the transportation channel so as to align them behind one another substantially in a hydrodynamic focusing plane or substantially along a hydrodynamic focusing axis, and the distance, between the outlet into the transportation channel of the through passage for the fluorescence excitation and said hydrodynamic focusing plane or said hydrodynamic focusing axis of the eukaryotic cells in the transportation channel, is less than 1 mm, more preferably less than 100 μm, and still more preferably less than 50 μm.
the eukaryotic cells are hydrodynamically focused in the transportation channel so as to align them behind one another substantially in a hydrodynamic focusing plane or substantially along a hydrodynamic focusing axis, and the distance between the outlet of the transportation channel of the through passage for the fluorescence detection and the hydrodynamic focusing plane or the hydrodynamic focusing axis of the eukaryotic cells in the transportation channel is less than 1 mm, more preferably less than 100 μm, and still more preferably less than 50 μm.
the eukaryotic cells are sperm cells (SP), in particular animal sperm cells, being able to have different chromosomes.

The invention also relates to sex-selected semen obtained by carrying out the aforementioned method allowing the in vitro selection of sperm cells.

### BRIEF DESCRIPTION OF THE FIGURES

The features and advantages of the invention will appear more clearly upon reading the detailed description below of several alternative embodiments of the invention, the description being provided as a non-limiting and non-exhaustive example of the invention, and in reference to the appended drawings, in which:

FIG. 1 is a schematic illustration of a first alternative embodiment of a selection device according to the invention, the microfluidic chip of the selection device being shown in top view;

FIG. 2 is a cross-sectional view of the microfluidic chip of the selection device, in cutting plane II-II of FIG. 1;

FIG. 3 is a cross-sectional view of this sorting device, in cutting plane III-III of FIG. 1;

FIG. 4 is a cross-sectional view of this sorting device, in cutting plane IV-IV of FIG. 3;

FIG. 5 is a cross-sectional view of this sorting device, in cutting plane V-V of FIG. 1;

FIG. 6 is a cross-sectional view of this sorting device, in cutting plane VI-VI of FIG. 1;

FIG. 7 is a cross-sectional view of this sorting device, in cutting plane VII-VII of FIG. 6;

FIG. 8 is a cross-sectional view of a second alternative of a selection device with optical fiber of the “wedge” type, in the same cutting plane as FIG. 7;

FIG. 9 shows the distal part of an optical fiber of the “wedge” type;

FIG. 10 is a cross-sectional view of a third alternative of a selection device with an optical fiber of the “tapered” type, in the same cutting plane as FIG. 7;

FIG. 11 is a schematic illustration of a fourth alternative embodiment of a sorting device according to the invention, the microfluidic chip of this selection device being shown in top view;

FIG. 12 is a cross-sectional view of the microfluidic chip of a fifth alternative of a selection device.

### DETAILED DESCRIPTION OF THE INVENTION

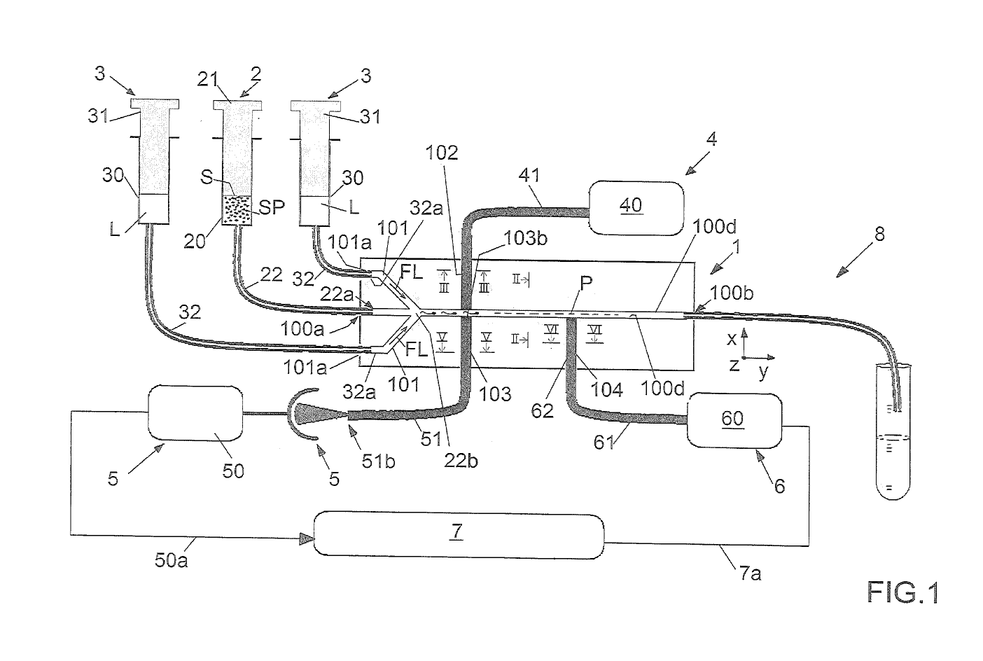

FIG. 1 shows a first alternative embodiment of a device allowing the selection of sperm cells based on their chromosome, and for example their X or Y chromosome.

The detailed description below is based on the selection of sperm cells differing by their chromosome type. The invention is not, however, limited to sperm cell selection, but may apply more generally to the selection of eukaryotic cells that may have different types making it possible to inventory them in at least two separate categories. More particularly, but not exclusively, the eukaryotic cells can for example differ owing to the DNA of their core.

The device of FIG. 1 includes:
a microfluidic chip 1 including a primary microfluidic transportation channel 100, injection means 2 making it possible to inject a solution S containing a sample of sperm cells SP to be selected into the primary microfluidic transportation channel 100,
hydrodynamic focusing means (3, 101) making it possible to inject a liquid L into the primary microfluidic transportation channel 100 so as to obtain a hydrodynamic focusing of the sperm cells in the primary microfluidic transportation channel 100,
excitation means 4 of the fluorescence of the sperm cells SP circulating in the primary microfluidic channel 100,
means 5 for detecting the fluorescence emitted by the sperm cells circulating in the primary microfluidic transportation channel 100,
selection means 6, including a source of electromagnetic radiation 60, and making it possible to selectively alter the sperm cells circulating in the primary microfluidic transportation channel 100, using the electromagnetic radiation emitted by the source 60,
electronic control means 7, which make it possible to control said source of electromagnetic radiation 60 automatically, from the fluorescence detection done by the detection means 5,
collection means 8 making it possible to collect all of the sperm cells leaving the primary microfluidic transportation channel 100.

### Microfluidic Chip 1—Primary Microfluidic Transportation Channel 100

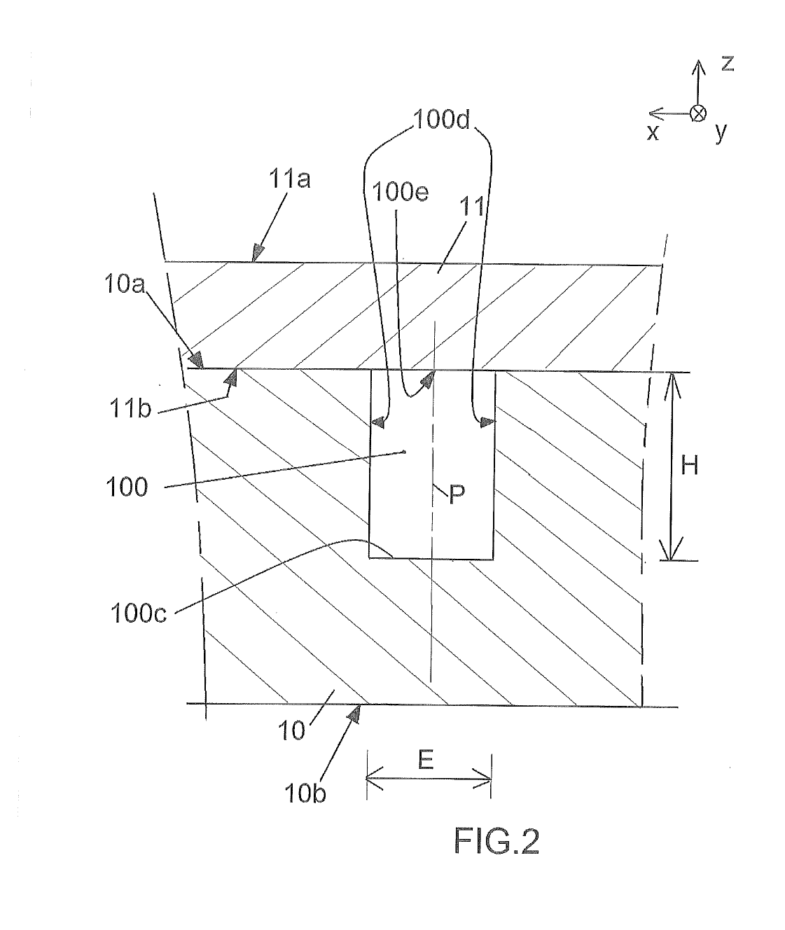

In reference to FIG. 2, the microfluidic chip 1 comprises a rigid substrate 10, having a front face 10a and a rear face 10b, and a plate 11 fastened against the front face 10a of the substrate 10.

In reference to FIG. 1, in this particular alternative embodiment, the primary microfluidic transportation channel 100 is straight and extends along the longitudinal direction Y corresponding to the movement direction of the sperm cells in the microfluidic channel 100. This microfluidic channel 100 traverses the substrate 10 and includes an intake opening 100a and discharge opening 100b.

In another alternative, all or part of this primary microfluidic channel 100 may not be straight.

More particularly, in reference to FIG. 2, the cross-section (in a plane (X, Z)) of the primary microfluidic transportation channel 100 is rectangular, with a height H measured in the direction Z perpendicular to the planar upper face 10a of the substrate 10, and with a width E measured in the transverse direction X, parallel to the planar upper face 10a of the substrate 10 and perpendicular to the longitudinal direction Y of the microfluidic channel. The height H of the channel 100 is greater than the width E of the microfluidic channel 100.

Preferably, the width E of the channel is smaller than 1 mm, and more preferably still, smaller than 100 μm. The height H of the channel can also be smaller than 1 mm.

The structure and manufacturing technique of the microfluidic chip 1 including said microfluidic transportation channel 100 are unimportant and not limiting with respect to the invention.

Purely as an example, in the alternative embodiment of FIG. 2, the primary microfluidic channel 100 is defined by a slot having a U shape in cross-section and made in the upper face 10a of the substrate 10, and by the rear face 11b of the plate 11. The bottom wall 100c of the slot forms the bottom wall of the microfluidic channel 100; the two walls 100d of the slot that are parallel, and that are transverse, and more particularly perpendicular to the bottom wall 100c, form the two longitudinal walls 100d of the microfluidic channel 100; the part of the lower face 11b of the plate 11, situated at the U-shaped slot, forms the upper wall 100e of the microfluidic channel 100.

This rectangular cross-section of the microfluidic channel 100 makes it possible, in a manner known in itself, to facilitate the spatial orientation of the sperm cells in the channel 100 during the hydrodynamic focusing step. In particular, the sperm cells having a non-spherical and flattened shape, the implantation of a microfluidic channel 100 with a rectangular cross-section contributes to a spatial orientation of the sperm cells with their flattened face with a larger surface oriented substantially parallel to the plane (Y, Z), i.e., substantially parallel to the plane of the two longitudinal walls 100d of the microfluidic channel 100. One skilled in the art is responsible for carefully selecting the dimensions of the microfluidic channel 100, and in particular the ratio H/E, in a known manner.

It should, however, be stressed that the invention is not limited to the implementation of a transportation channel 100 having a rectangular cross-section, said transportation channel 100 more generally being able to have a different geometry in cross-section, and for example, non-limitingly and non-exhaustively being able to have a circular, oval, polygonal shape.

The substrate 10 is made from a material that is chemically inert. For example, but not necessarily, the material of the substrate 10 is chosen so as to be able to undergo physical or chemical etching, for example plasma etching. More particularly, but non-limitingly with respect to the invention, the substrate is for example made from silicon or gallium arsenide. In this case, the slot (100c, 100d) with height H and width E is advantageously made by anisotropic etching of the upper face 10a of the substrate 10. The substrate with the slot (100c, 100d) can also be produced by 3D printing.

The plate 11 is made from a material that is chemically inert, and may be opaque or transparent. The plate 11 is for example made from glass or plastic. It is fixed to the substrate 10 using any means, and for example by anode adhesion or thermocompression.

### Injection Means 2 for Injecting Sperm Cells into the Microfluidic Channel

The purpose of the injection means 2 is to inject a solution S containing a sample of sperm cells to be selected into the primary microfluidic channel 100.

More particularly, in the alternative of FIG. 1, these injection means 2 include a syringe 20, which is filled with a solution S containing the sample of sperm cells SP to be selected, and which is associated with an automatic injection system 21, which may for example be of the syringe plunger or peristaltic pump type. The outlet of the syringe 20 is coupled to a capillary tube 22, the distal part 22a of which has been inserted into the microfluidic channel 100 through the intake opening 100a of this channel 100. The capillary tube 22 is a flexible tube, the cross-section of which is preferably adapted to the section of the microfluidic channel 100.

More particularly, the capillary tube 22 is preferably fixed to the substrate 10 using any means, and in particular by adhesion.

The sperm cells SP contained in the syringe 20 are diluted in the buffer solution S, which is biologically compatible with the sperm cells, and for example in an aqueous solution including 30 g/L of TRIS (trishydroxymethylaminomethane), 17.25 g/L of monohydrate citric acid, and 12.5 g/L of fructose in water at a pH of about 7. Many other buffer solutions S known by those skilled in the art can be used. On this point, reference may for example be made to the teaching of international patent application WO2004/088283.

More particularly, the DNA of the sperm cells SP contained in the buffer solution S has been marked, in a manner known in itself, using at least one fluorochrome, which can fluoresce when it is associated with DNA. Among the fluorochromes commonly used to mark sperm cells, non-limiting and non-exhaustive examples include: fluorochromes of the bisbenzimide type, and in particular Hoechst fluorochromes (Hoechst 33342, Hoechst 33258, etc.), ethidium bromide, SYBR fluorochromes such as SYBR-14.

Many other fluorochromes known by those skilled in the art can be used. In particular, for more ample details on producing a buffer solution S including sperm cells marked using fluorochromes, reference may for example be made to the teaching of international patent application WO2004/088283.

During operation, the injection system 21 pushes the buffer solution S containing the sperm cells SP, so as to cause it to leave through the distal opening 22b of the capillary 22, and to inject it into the microfluidic channel 100 with an automatically controlled flow rate, which is preferably constant. It has been possible to verify that this injection of the buffer solution S containing the sperm cells SP into the microfluidic channel 100 did not affect fertility, and in particular the motility of sperm cells.

### Means (3, 101) for Hydrodynamic Focusing of the Sperm Cells

In order to allow the implementation of hydrodynamic focusing of the sperm cells SP in the microfluidic channel 100, the microfluidic chip 1 includes two secondary microfluidic channels 101, which are typically formed on either side of the microfluidic transportation channel 100 (FIG. 1). Each secondary microfluidic channel 101 includes an intake opening 101a and emerges, opposite the intake opening 101a, laterally in the primary microfluidic channel 100.

The outlet of the capillary tube 22 is positioned upstream from the junction zone between the secondary microfluidic channels 101 and the microfluidic transportation channel 100, the distal part 22a of the capillary tube 22 being able to be inserted more or less deeply into the transportation channel 100.

More particularly, and similarly to what was previously described for the primary microfluidic channel 100, each secondary microfluidic channel 101 is defined on the one hand by a U-shaped slot etched in the upper face of the substrate 10, and on the other hand by the lower face 11b of the plate 11.

The hydrodynamic focusing means include, for each secondary channel 101, injection means 3 in the form of a syringe 30, which is filled with a solution L, and which is associated with an automatic injection system 31, for example of the syringe plunger or peristaltic pump type. The outlet of the syringe 30 is coupled to a capillary tube 32, the distal part 32a of which has been inserted into the microfluidic channel 100 through the intake opening 101a of a secondary channel 101. Each capillary tube 32 is a flexible tube, the cross-section of which is preferably adapted to the section of the secondary channel 101. More particularly, each capillary tube 32 is preferably fixed to the substrate 10, using any means, and in particular by adhesion.

The liquid used for the solutions L is preferably, but not necessarily, identical to that used for the buffer solution S containing the sperm cells SP.

During operation, each injection system 31 pushes each solution L so as to inject it into the corresponding secondary microfluidic channel 101 with a flow rate controlled automatically, and that is preferably constant.

In a manner known in itself, the flow rates of each solution L and the buffer solution S containing the sperm cells SP are checked automatically, so as to create two laminar flows FL in the microfluidic channel 100a with a high speed that are formed by each solution L, on either side of the central flow, which is slower, formed by the solution S containing the sperm cells SP. These laminar flows FL make it possible, in a known manner, to drive the sperm cells SP in the primary microfluidic channel 100 by causing them to undergo hydrodynamic focusing, of the 2D type, which substantially results in aligning the sperm cells SP behind one another, with a substantially constant spacing between two adjacent sperm cells, and with an alignment of the sperm cells SP substantially in a longitudinal plane P parallel to the plane (Y, Z).

The position of this hydrodynamic focusing plane P of the sperm cells between the two longitudinal walls 100d of the channel 100 in particular depends on the difference in speed between the two laminar flows FL of liquid L. When the speeds are equal, the hydrodynamic focusing plane P of the sperm cells is substantially centered between the two longitudinal walls 100d of the channel 100 (FIG. 2). In the context of the invention, the hydrodynamic focusing plane P of the sperm cells can be off-centered relative to the two longitudinal walls 100d of the channel 100.

The invention is not limited to a 2D hydrodynamic focusing of the sperm cells SP in the primary microfluidic channel 100. It is also possible, in the context of the invention, to carry out 3D hydrodynamic focusing, as for example described in international patent application WO2011/005776, so as to align the sperm cells substantially along the longitudinal hydrodynamic focusing axis parallel to the axis Y of the microfluidic channel 100.

### Fluorescence Excitation Means 4

The fluorescence excitation means 4 include an electrostatic radiation source 40, of the laser source type, the wavelength of which is adapted to the marker (fluorochrome) of the sperm cells. For example, and non-limitingly with respect to the invention, an electromagnetic excitation radiation is used with a wavelength comprised between 300 nm and 400 nm, and for example more particularly around 375 nm when the sperm cells SP have been marked with Hoechst.

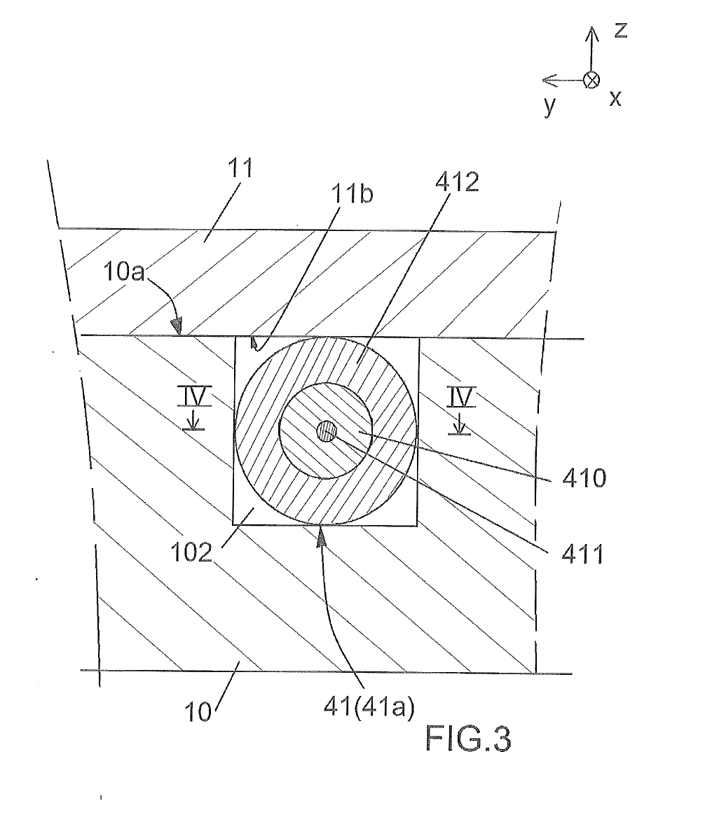

The microfluidic chip 1 includes a first through passage 102, which opens into the primary microfluidic channel 100 (FIGS. 1 and 3) in a part of the primary microfluidic channel 100 situated downstream from the hydrodynamic focusing zone (junction zone between the channels 100 and 101).

In the specific alternative of FIG. 1, this through passage 102 is made through one of the longitudinal walls 100d of the microfluidic channel 100.

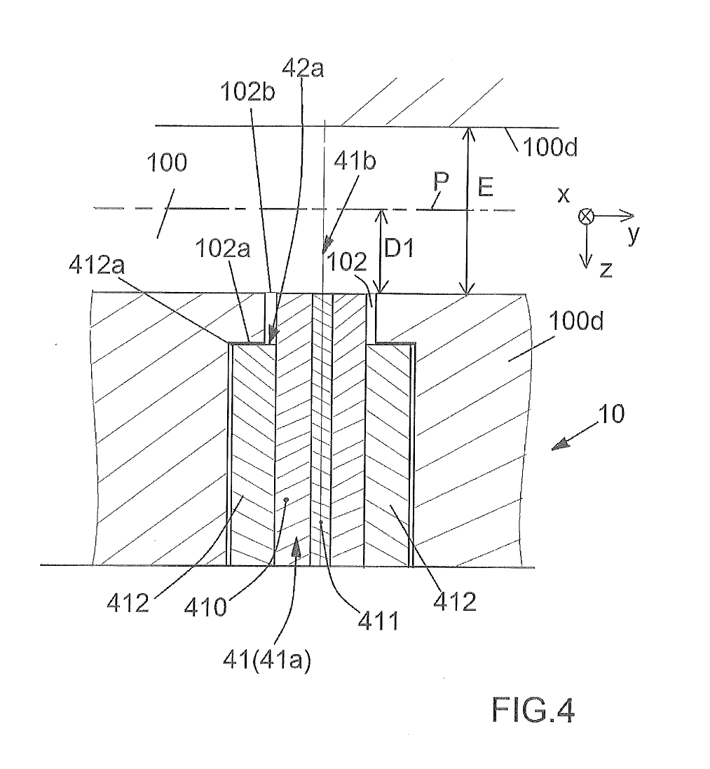

In the alternative illustrated in FIGS. 1 and 4, the distance between the outlet 102b in the transportation channel 100 of the through passage 102 and the opposite wall 100d of the transportation channel 100 corresponds to the width E of the transportation channel 100, and is preferably smaller than 1 mm, more preferably smaller than 100 μm.

This through passage 102 is defined by a U-shaped slot etched in the upper face 10a of the substrate 10 and by the lower face 11b of the plate 11. In another alternative, the through passage 101 could be pierced through the substrate 10.

The source of the electromagnetic radiation 40 is coupled to an optical fiber 41, the distal part 41a of which is inserted in this first through passage 102, such that the emission end 41b (FIG. 4) of the optical fiber 41 does not protrude in the microfluidic channel 100, so as not to disrupt the flows of fluids in the microfluidic channel 100, and thus not disrupt the positioning and spatial orientation of the sperm cells SP circulating in the microfluidic channel 100. Preferably, the emission end 41b of the optical fiber 41 is positioned as close as possible to this microfluidic channel 100, and is preferably flush with the microfluidic channel 100.

In this FIG. 4, the mechanical sheath of the optical fiber is referenced 412, the optical sheath of the optical fiber is referenced 410, and the core of the optical fiber is referenced 411. In this alternative, the distal end of the optical sheath 410 and the core 411 of the optical fiber, by which the electromagnetic excitation radiation is emitted, is stripped. In another alternative, the distal end of the optical sheath 410 and the core 411 of the optical fiber could not be stripped and be surrounded by the mechanical sheath 412 of the optical fiber 41.

More particularly, in the alternative of FIG. 4, the through passage 102 is profiled so as to include a shoulder 102a forming a positioning stop 102a for the distal end 412a of the mechanical sheath 412 of the optical fiber. It suffices to insert the optical fiber until the distal end 412a of the mechanical sheath 412 is blocked by the positioning stop 102a, which advantageously allows simple and precise positioning of the distal emission end of the optical fiber 41 relative to the microfluidic channel 100.

During operation, when the emission end of the optical fiber 41 is flush with the transportation channel 100, the electromagnetic excitation radiation is emitted by the optical fiber 41 directly in the transportation channel 100. When the emission end of the optical fiber 41 is positioned slightly withdrawn in the through passage 102, the electromagnetic excitation radiation is emitted in this through passage 102, then penetrates the transportation channel 100.

This insertion of the optical fiber 41 into the microfluidic chip 1, near the microfluidic channel 100, advantageously makes it possible to bring the electromagnetic excitation radiation as close as possible to the sperm cells SP circulating in the microfluidic channel, which contributes to improving the performance of the excitation of the fluorescence.

So as also to improve the performance of the excitation of the fluorescence, the distance D₁ (FIG. 4), between the outlet 102b in the transportation channel 100 of the first through passage 102 and the hydrodynamic focusing plane P (2D hydrodynamic focusing) or the hydrodynamic focusing axis (3D hydrodynamic focusing) of the sperm cells SP in the transportation channel 100, is preferably very small. One thereby advantageously reduces the length of the journey of the electromagnetic excitation radiation to the sperm cells SP, through the liquid transporting the sperm cells in the transportation channel 100. Preferably, but not necessarily, this distance D₁ is less than 1 mm, more preferably less than 100 μm, and still more preferably less than 50 μm.

The insertion of the optical fiber 41 into the microfluidic chip 1 also makes it possible to avoid the risks of misalignment of the electromagnetic excitation radiation relative to the microfluidic channel 100 when the microfluidic chip 1 is manipulated.

### Fluorescence Detection Means 5

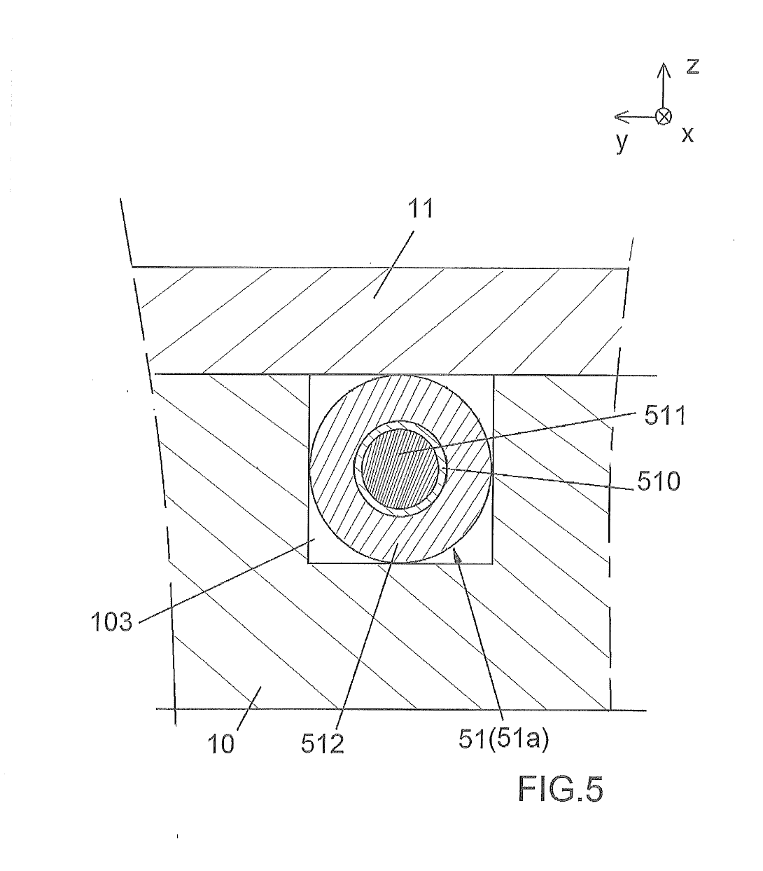

The fluorescence detection means 5 include an optical fiber 51 and a photodetector 50, of the photomultiplier (PM) type, that is adapted for detecting the fluorescence wavelength of the sperm cells, i.e., for example, a wavelength comprised between 400 nm and 500 nm, and for example around 460 nm when the sperm cells have been marked with a fluorochrome of the Hoechst type. [0078]The microfluidic chip 1 includes a second through passage 103, which opens into the primary microfluidic channel 100 (FIGS. 1 and 5) across from the first through passage 103. In the particular alternative of FIG. 1, this through passage 103 is made through the other longitudinal wall 100d of the microfluidic channel 100.

The distance between the outlet 103b in the transportation channel 100 of the through passage 103 and the opposite wall 100d of the transportation channel 100 corresponds to the width E of the transportation channel 100, and is preferably less than 1 mm, more preferably less than 100 μm.

In this FIG. 5, the mechanical sheath of the optical fiber is referenced 512, the optical sheath of the optical fiber is referenced 510, and the core of the optical fiber is referenced 511.

Identically to what was described previously for the optical fiber 41, the distal part 51a of the optical detection fiber 51 is inserted into this second through passage 103 such that the distal end of the optical fiber 51 does not protrude in the microfluidic channel 100, and is preferably positioned as close as possible to this microfluidic channel 100.

The emission end 51b of the detection fiber 51 is positioned across from the photodetector 50, such that the light (fluorescence) that is emitted in the microfluidic channel 100, and which is captured by the fiber 51, is detected by the light detector 50. The light detector 50 delivers an electric signal 50a characteristic of the light intensity of the fluorescence that is detected.

This insertion of the optical detection fiber 51 and the microfluidic chip 1, near the microfluidic channel 100, advantageously makes it possible to improve the fluorescence detection and contributes to better discrimination between an X sperm cell and a Y sperm cell.

In order also to improve the performance of the fluorescence detection, the distance between the outlet 103b in the transportation channel 100 of the second through passage 103 (FIG. 1—opening 103b of this passage 103 opening in the transportation channel 100) and the hydrodynamic focusing plane P (2D hydrodynamic focusing) or the hydrodynamic focusing axis (3D hydrodynamic focusing) of the sperm cells in the transportation channel 100 is preferably very small. The length of the journey of the fluorescence radiation is thus advantageously reduced, through the liquid transporting the sperm cells in the transportation channel 100. Preferably, but not necessarily, this distance is smaller than 1 mm, preferably smaller than 100 μm, and still more preferably smaller than 50 μm.

More particularly, in order to collect a maximum amount of light, the optical detection fiber 51 can for example be a large core optical fiber 511 (FIG. 5), unlike the optical excitation fiber 41, the core 411 of which (FIG. 4) has a smaller diameter so as to spatially concentrate the electromagnetic radiation.

### Selection Means 6

The selection means 6 include a source 60 of electromagnetic radiation, of the laser source type (pulsed or continuous), and an optical fiber 61. The output of this source of electromagnetic radiation 61 is coupled to the optical fiber 61.

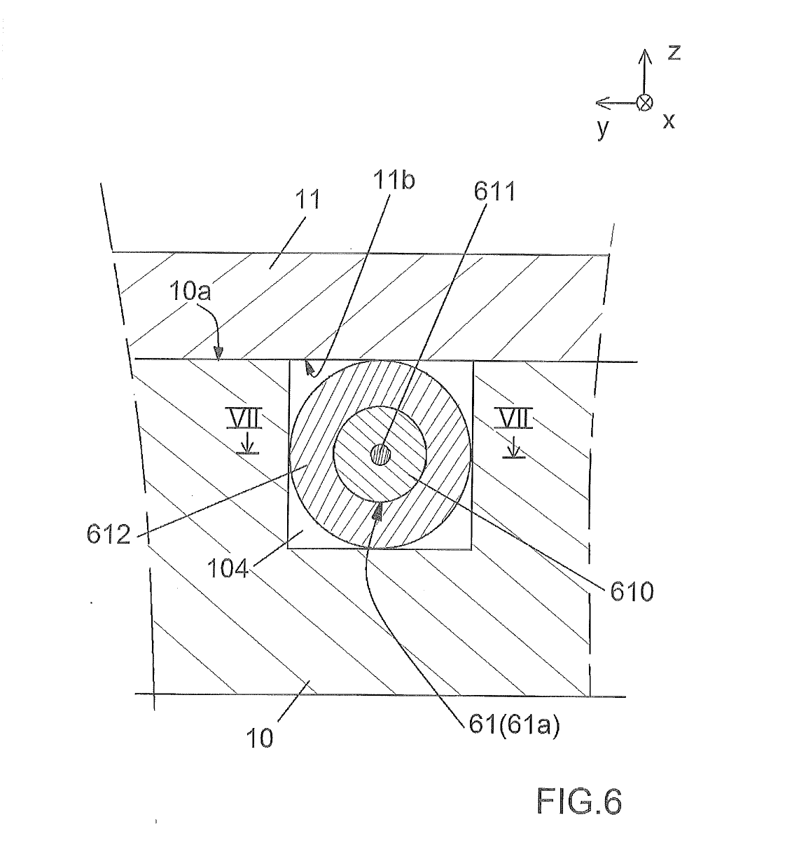

The microfluidic chip 1 includes a third through passage 104, which opens into the primary microfluidic channel 100 (FIGS. 1 and 6) in part of the primary microfluidic channel 100 situated downstream from the fluorescence detection zone.

In the specific embodiment of FIG. 1, this through passage 104 is made through one of the longitudinal walls 100d of the microfluidic channel 100.

The distance between the outlet 104b in the transportation channel 100 of the through passage 104 and the opposite wall 100d of the transportation channel 100 corresponds to the width E of the transportation channel 100, and is preferably smaller than 1 mm, more preferably smaller than 100 μm.

In FIG. 6, the mechanical sheath of the optical fiber is referenced 612, the optical sheath of the optical fiber is referenced 610, and the core of the optical fiber is referenced 611.

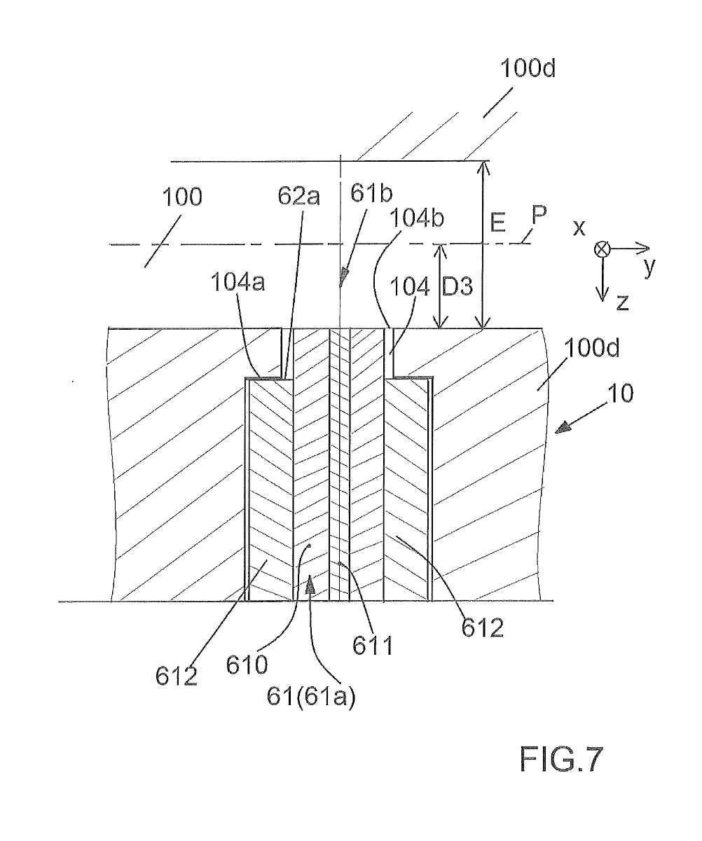

Identically to what was described previously for the optical fiber 41, the distal part 61a of the optical fiber 61 is inserted into this third through passage 104, such that the distal emission end 61b (FIG. 7) of the optical fiber 61 does not protrude in the microfluidic channel 100, and is preferably positioned as close as possible to this microfluidic channel 100.

When it is activated, the source 60 emits, in the microfluidic channel 100, an electromagnetic alteration radiation R, the purpose of which is to alter, directly or indirectly, a sperm cell SP circulating in the primary microfluidic channel 100 and passing through this electromagnetic alteration radiation, such that this sperm cell is no longer fertile. The sperm cell SP traversing the electromagnetic alteration radiation is modified, such that its viability or motility is deteriorated enough for the sperm cell no longer to be fertile, independently of the physical and/or biological and/or chemical phenomenon causing this deterioration. In the general context of the invention, this alteration by the electromagnetic radiation may for example result from an injury or photochemical alteration of the sperm cell and/or a thermal stress effect experienced by the sperm cell.

To obtain this alteration, it is possible to use a wavelength for the electromagnetic alteration radiation that is selected in a wide spectral range. More particularly, but not exclusively, the wavelength of the electromagnetic alteration radiation will typically be selected from a wavelength range going from UV to Infrared.

The insertion of the optical fiber 61 into the microfluidic chip 1, near the microfluidic channel 100, advantageously makes it possible to bring the electromagnetic alteration radiation of the sperm cells as close as possible to the sperm cells SP circulating in the microfluidic channel.

During operation, when the emission end of the optical fiber 61 is flush with the transportation channel 100, the electromagnetic alteration radiation is emitted by the optical fiber 61 directly in the transportation channel 100. When the emission end of the optical fiber 61 is positioned slightly withdrawn in the through passage 104, the electromagnetic alteration radiation is emitted in this through passage 102, then penetrates the transportation channel 100.

In order also to improve the effects of the electromagnetic alteration radiation, the distance D₃ (FIG. 7), between the outlet 104b in the transportation channel 100 of the third through passage 104 and the hydrodynamic focusing plane P (2D hydrodynamic focusing) or the hydrodynamic focusing axis (3D hydrodynamic focusing) of the sperm cells SP in the transportation channel 100, is preferably very small. One thus advantageously reduces the length of the journey of the electromagnetic alteration radiation to the sperm cells SP, through the liquid transporting the sperm cells in the transportation channel 100. Preferably, but not necessarily, this distance D₃ is smaller than 1 mm, more preferably smaller than 100 μm, and still more preferably smaller than 50 μm.

This in particular results in a considerable reduction in the absorption phenomena of the electromagnetic alteration radiation by the liquid circulating in the microfluidic channel 100, and the absorption phenomena of the electromagnetic alteration radiation by the substrate 10 of the microfluidic chip are eliminated, compared to a solution in which the electromagnetic alteration radiation must pass through said substrate. It thus becomes possible to alter the sperm cells by irradiating them with a low-power electromagnetic radiation, for example having a mean power of less than 10 W, preferably less than 1 W. The invention is not, however, limited to these power values.

Owing to the integration of the distal part 61a of the optical fiber 61 into the microfluidic chip, one also avoids the risk of misalignment of the electromagnetic alteration radiation relative to the microfluidic channel 100, in particular when the microfluidic chip 1 is manipulated.

### Focusing—“Wedge” Fiber or “Tapered” Fiber

Preferably, the optical fiber 61 is a micro-lensed optical fiber whereof the emission end 61b is profiled so as to form a lens making it possible to focus the electromagnetic alteration radiation R in the microfluidic channel 100 relative to the journey of the sperm cells SP, i.e., in the case of the appended figures, in the alignment plane P of the sperm cells SP.

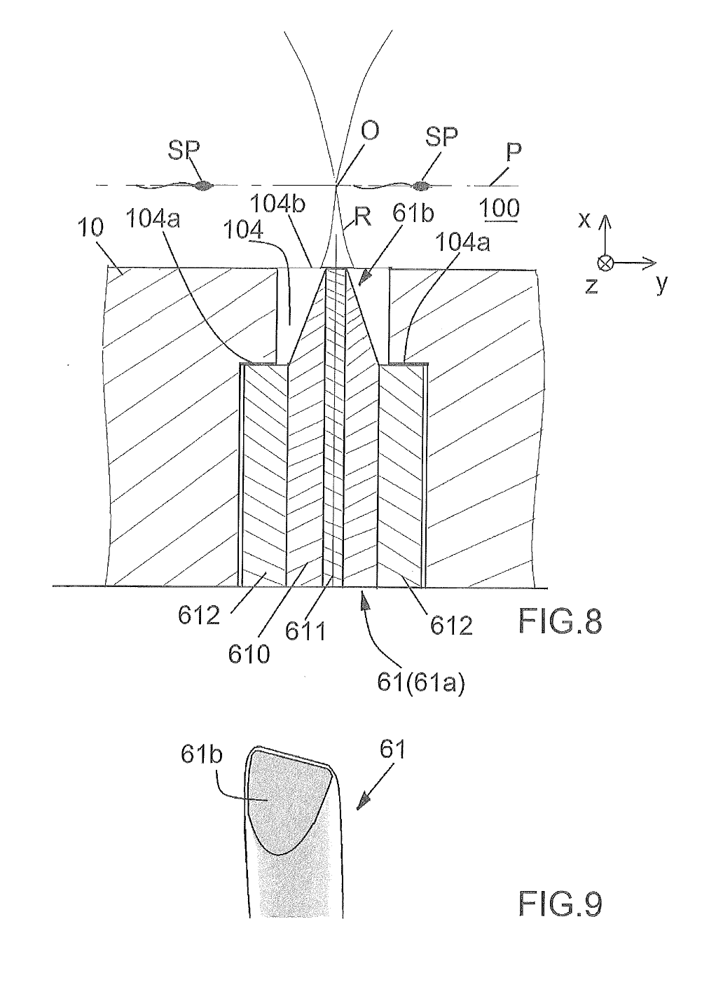

More particularly, in reference to FIGS. 8 and 9, in an alternative embodiment, the optical fiber 61 is a so-called “wedge” optical fiber, i.e., whereof the distal emission end forms a beveled lens on both faces in one direction.

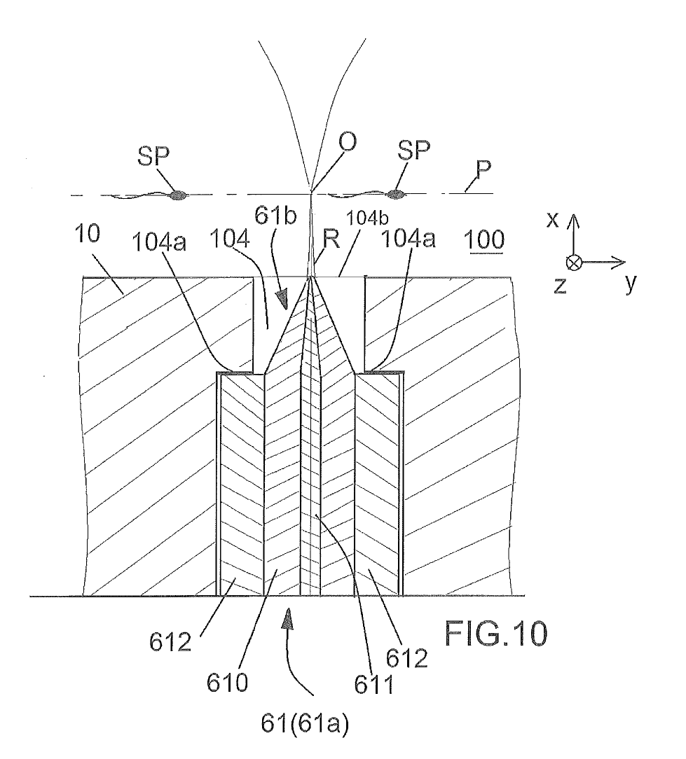

More particularly, in reference to FIG. 10 in one alternative embodiment, the optical fiber 61 is a so-called “tapered” optical fiber, i.e., whereof the distal emission end forms a conical lens.

The position of the emission end 61b of the optical fiber 61 relative to the hydrodynamic focusing plane P of the sperm cells SP is chosen so as to optimize the interaction between the electromagnetic alteration radiation F relative to the hydrodynamic focusing plane P of the sperm cells SP, so as to deliver the maximum energy substantially in this alignment plane P of the sperm cells.

More particularly, in reference to FIG. 8, the “wedge” optical fiber 61 makes it possible to focus the electromagnetic alteration radiation R substantially along a focusing line O in the hydrodynamic focusing plane P of the sperm cells SP and parallel to the axis Z. The “tapered” optical fiber allows focusing of the electromagnetic alteration radiation R at a focusing point O substantially in the hydrodynamic focusing plane P of the sperm cells SP.

One thus optimizes the use of the power of the electromagnetic alteration radiation R to obtain the alteration of the sperm cells SP, which makes it possible to reduce the power of the electromagnetic radiation source 60.

### Electronic Control Means 7

The electronic control means 7 receive, as input, the fluorescence detection signal 50a delivered by the photodetector 50, and as output, deliver a control signal 7a making it possible to control said electromagnetic radiation source 60 automatically, from the fluorescence detection.

More particularly, the electronic control means 7 are for example designed to compare the fluorescence detection signal 50a with a predefined threshold, which, in a known manner, makes it possible to discriminate between an X sperm cell and a Y sperm cell, and to automatically control said electromagnetic radiation source 60 such that:
the electromagnetic alteration radiation R is emitted in the microfluidic channel 100, if one wishes to alter the sperm cell SP that has been detected when the latter traverses said electromagnetic alteration radiation R; or
the electromagnetic alteration radiation R is not emitted in the microfluidic channel 100, if one wishes to keep the sperm cell SP that has been detected intact.

In the sample collected using the collection means 8, one thus obtains a sex-selected semen; the sperm cells of one given type (for example, Y) are thus intact and fertile, and the sperm cells of the other type (for example, X) are altered enough to no longer be fertile.

The invention in particular making it possible to reduce the power of the electromagnetic radiation source, the modulation of the electromagnetic alteration radiation R based on the fluorescence detection can be very fast, which makes it possible to achieve high selection rhythms.

Solely as an example, and non-limitingly, it is for example possible to implement the invention with a power laser source 60 of about several hundred mW, emitting in a wavelength range between 1 μm and 3 μm, and with a “tapered” optical fiber 60, and to select sperm cells with a rhythm of one sperm cell every 100 μs.

### Other Alternatives

The invention is not limited to the aforementioned alternative embodiments. Non-limitingly and non-exhaustively with respect to the invention, other alternative embodiments briefly described below may for example be considered.

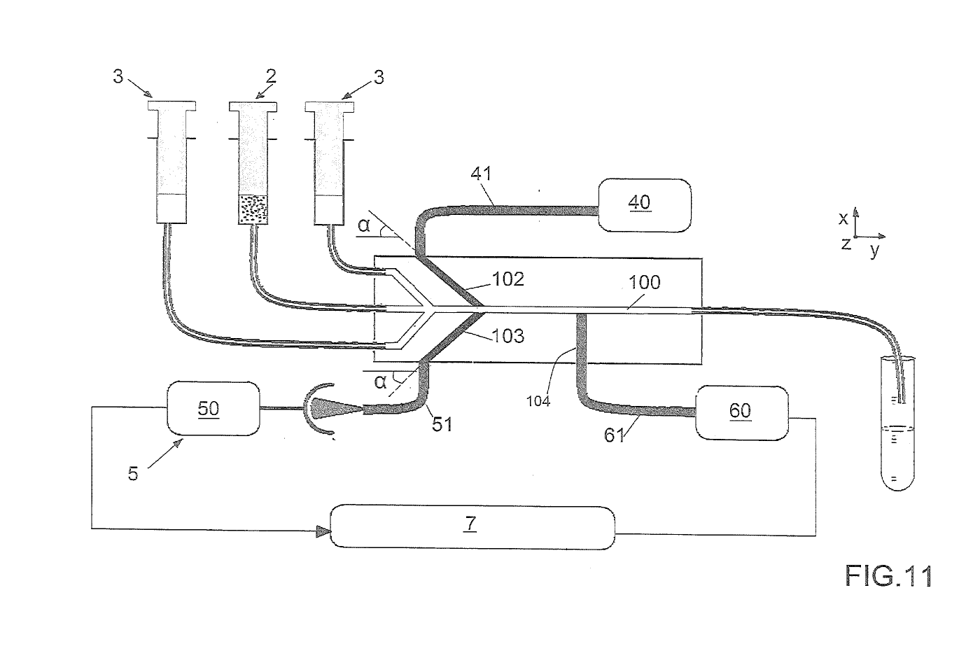

In reference to the alternative of FIG. 11, the through passages 102 and 103 for the optical excitation fiber 41 and the optical fluorescence detection fiber 51, respectively, are not necessarily oriented perpendicular to the longitudinal axis Y of the primary microfluidic channel 100, but can form an angle α of less than 90° with this longitudinal axis Y.

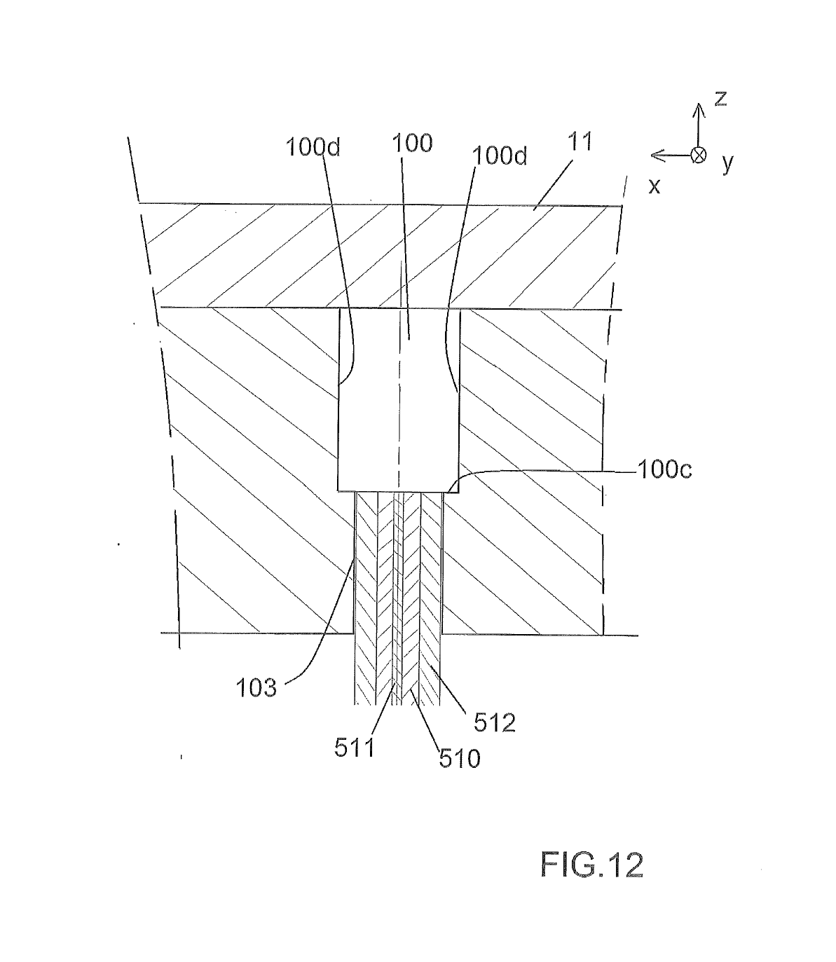

In reference to the alternative of FIG. 12, the optical fiber 51 for detecting the fluorescence can be integrated into the microfluidic chip 11 having its distal part inserted into a through passage 103 made in the bottom wall 100c of the microfluidic channel 100. The optical fibers 41 and 51 are thus advantageously oriented at a right angle relative to one another, which makes it possible to avoid the risks of detection of a stray fluorescence liquid that could in particular be emitted by the optical fiber 41 of the fluorescence excitation means 4. Conversely, in another alternative, it may be the optical fiber 41 exciting the fluorescence that can be integrated into the microfluidic chip 1 by having its distal part inserted into a through passage 103 made in the bottom wall 100c of the microfluidic channel 100.

In another alternative embodiment (not shown), the fluorescence excitation means 4 may be completely outside the microfluidic chip 1 and not include an optical fiber integrated into the microfluidic chip 1.

Likewise, the fluorescence detection means 5 can be completely outside the microfluidic chip 1 and not include an optical fiber integrated into the microfluidic chip 1.

1. A device for selecting eukaryotic cells (SP), said device including a transportation channel (100), in which a solution containing said eukaryotic cells (SP) can circulate, a first through passage (104) opening into said transportation channel (100), a source of electromagnetic radiation (60), which is coupled to a first end of a first optical fiber (61), the other emission end (61b) of the first optical fiber being inserted into said first through passage (104), without protruding into the transportation channel (100), said device further including electronic control means (7), for controling said source of electromagnetic radiation (60) automatically, so as to selectively alter the eukaryotic cells (SP) circulating in the transportation channel (100), by means of the electromagnetic alteration radiation (R) emitted by the source of electromagnetic radiation (60).

2. The device according to claim 1, wherein the transportation channel (100) is a microfluidic channel whereof at least one dimension in cross-section is smaller than 1 mm.

3. The device according to claim 1, including optical focusing means, which are fixed or integrated to the emission end (61b) of the first optical fiber, and which make it possible to focus, in said transportation channel (100), the electromagnetic radiation (R) emitted at the outlet of the first optical fiber (61).

4. The device according to claim 3, wherein said optical focusing means are formed by the emission end (61b) of the first optical fiber (61), which is profiled so as to focus, in said transportation channel (100), the electromechanical radiation (R) emitted at the outlet of the optical fiber (61).

5. The device according to claim 4, wherein the emission end (61b) of the first optical fiber (61) has a conical shape or is of the “wedge” type.

6. (canceled)

7. The device according to claim 1, wherein the emission end (61b) of the first optical fiber is flush with the transportation channel (100) without protruding in the transportation channel.

8. The device according to claim 1, wherein the distal emission part (61a) of the first optical fiber (61) is inserted into said first through passage (104), abutting against a shoulder (104a).

9. The device according to claim 1, wherein the distal emission part (61a) of the first optical fiber (61) is inserted into said first through passage (104), abutting against a shoulder (104a), wherein the transportation channel (100) has a rectangular cross-section.

10. (canceled)

11. The device according to claim 9, wherein the first through passage (104) is made through one of the longitudinal walls (100d) with a larger dimension (H) of the transportation channel (100) with a rectangular section.

12. The device according to claim 1, including a second source of electromagnetic radiation (40) able to emit electromagnetic excitation radiation, which makes it possible to excite the emission by fluorescence of the eukaryotic cells (SP) circulating in said transportation channel (100), and at least one photodetector (50) making it possible to detect the fluorescence emitted by said eukaryotic cells (SP).

13. The device according to claim 12, wherein the electronic control means (7) are able to process an electrical detection signal (50a) delivered by said photodetector (50), and to control the first source of electromagnetic radiation (60), for the selective alteration of the eukaryotic cells (SP), based on this detection signal (50a).

14. The device according to claim 12, including a through passage (102) for the fluorescence excitation, which opens into said transportation channel (100), upstream of the first through passage (104), and wherein the second source of electromagnetic radiation (40) is coupled to a first end of a fluorescence excitation optical fiber (41), the other emission end (41b) of this fluorescence excitation optical fiber (41) being inserted into said through passage (102) for the fluorescence excitation, without protruding in the transportation channel (100).

15. The device according to claim 14, wherein the distal emission part (41a) of the fluorescence excitation optical fiber (41) is inserted into said through passage (102) for the fluorescence excitation, abutting against a shoulder (102a).

16. The device according to claim 12, including a through passage (103) for the fluorescence detection, which opens into said transportation channel (100) upstream of the first through passage (104), and wherein one end (41b) of an optical fluorescence detection fiber (51) is inserted into this through passage (103), without protruding in the transportation channel (100), and the other emission end (51b) of said optical fluorescence detection fiber (51) being associated with the photodetector (50).

17. The device according to claim 16, wherein the optical fluorescence detection fiber (51) is a large core optical fiber.

18. The device according to claim 16, wherein a distal part of the optical fluorescence detection fiber (51) is inserted into said through passage (103) for the fluorescence detection, abutting against a shoulder (103a).

19. The device according to claim 14, wherein the transportation channel is defined at least by a bottom wall (100c) and by two longitudinal walls (100d) opposite one another that are transverse, to the bottom wall (100c), the through passage (102) for the fluorescence excitation is made through the bottom wall (100c) of the transportation channel (100), and the through passage (103) for the fluorescence detection is made through one of the longitudinal walls (100d) of the transportation channel (100) or the through passage (103) for the fluorescence detection is made through the bottom wall (100c) of the transportation channel (100), and the through passage (102) for the fluorescence excitation is made through one of the longitudinal walls (100d) of the transportation channel (100).

20. (canceled)

21. (canceled)

22. The device according to claim 14, wherein the distance (E) between the outlet (102b) in the transportation channel (100) of the through passage (102) for the fluorescence excitation and the opposite wall of the transportation channel (100) is less than 1 mm, preferably less than 100 μm.

23. The device according to claim 16, wherein the distance (E) between the outlet (103b) in the transportation channel (100) of the through passage (103) for the fluorescence detection and the opposite wall of the transportation channel (100) is smaller than 1 mm, preferably smaller than 100 μm.

24. The device according to claim 1, wherein the distance (E) between the outlet (104b) in the transportation channel (100) of the first through passage (104) and the opposite wall of the transportation channel (100) is less than 1 mm, preferably less than 100 μm, and still more preferably less than 50 μm.

25. The device according to claim 1, including means (2) making it possible to inject, in said transportation channel (100), a solution (S) containing eukaryotic cells (SP), and preferably eukaryotic cells (SP) marked using at least one fluorochrome.

26. The device according to claim 25, including hydrodynamic focusing means (3; 101) making it possible to inject a liquid (L) into the transportation channel (100) so as to drive the eukaryotic cells (SP) in the transportation channel (100), positioning them substantially in a hydrodynamic focusing plane (P) or substantially along a hydrodynamic focusing axis and spacing them apart one behind one another.

27. The device according to claim 26, wherein said optical focusing means make it possible to focus the electromagnetic alteration radiation (R) substantially in said hydrodynamic focusing plane (P) of the eukaryotic cells (SP) or substantially on the hydrodynamic focusing axis of the eukaryotic cells (SP).

28. The device according to claim 1, wherein the transportation channel (100) is defined in part by a slot etched in one (10a) of the faces of a hard substrate (10), and more particularly in a silicon substrate.

29. The device according to claim 28, wherein the first through passage (104), and if applicable the through passage (102) for the fluorescence excitation, and/or the through passage (103) for the fluorescence detection, are each defined in part by a slot edge in the same face (10a) of the substrate (10) as the transportation channel (100).

30. The device according to claim 1, wherein the mean power of the source (60) of electromagnetic alteration radiation of the eukaryotic cells (SP) is less than 10 W, and preferably less than 1 W.

31. (canceled)

32. A method for the in vitro selection of eukaryotic cells (SP) able to have different types making it possible to inventory them in at least two different categories, using a selection device as set out in claim 1, during which a solution (S) containing the eukaryotic cells (SP) to be sorted is injected into the transportation channel (100) of the sorting device; said eukaryotic cells (SP) are circulated in the transportation cell (100) one after another; the type of each eukaryotic cell circulating in the transportation channel (100) is detected automatically; and the eukaryotic cells (SP) that have been detected as being of the same predefined type are irradiated selectively, with the electromagnetic alteration radiation (R), so as to alter them enough to make them nonviable, the other eukaryotic cells not being altered using the electromagnetic alteration radiation (R).

33. The method according to claim 32, during which the eukaryotic cells are hydrodynamically focused in the transportation channel so as to align them behind one another substantially in a hydrodynamic focusing plane (P) or substantially along a hydrodynamic focusing axis, and wherein the distance (D₃) between the outlet (104b) into the transportation channel (100) of the first through passage (104) and the hydrodynamic focusing plane (P) or the hydrodynamic focusing axis of the eukaryotic cells (SP) in the transportation channel (100) is less than 1 mm, more preferably less than 100 μm, and still more preferably less than 50 μm.

34. The method according to claim 32, during which the eukaryotic cells are hydrodynamically focused in the transportation channel so as to align them behind one another substantially in a hydrodynamic focusing plane (P) or substantially along a hydrodynamic focusing axis, and wherein the distance (Di), between the outlet (102b) into the transportation channel (100) of the through passage (102) for the fluorescence excitation and said hydrodynamic focusing plane (P) or said hydrodynamic focusing axis of the eukaryotic cells (SP) in the transportation channel (100), is less than 1 mm, more preferably less than 100 μm, and still more preferably less than 50 μm.

35. The method according to claim 32, during which the eukaryotic cells are hydrodynamically focused in the transportation channel so as to align them behind one another substantially in a hydrodynamic focusing plane (P) or substantially along a hydrodynamic focusing axis, and wherein the distance between the outlet (103b) in the transportation channel (100) of the through passage (103) for the fluorescence detection and the hydrodynamic focusing plane (P) or the hydrodynamic focusing axis of the eukaryotic cells (SP) in the transportation channel (100), is less than 1 mm, more preferably less than 100 μm, and still more preferably less than 50 μm.

36. The method according to claim 32, wherein the eukaryotic cells are sperm cells (SP), in particular animal sperm cells, being able to have different chromosomes.

37. Sex-selected semen obtained by carrying out the method according to claim 36.

38. The device according to claim 6, wherein the distal emission part (61a) of the first optical fiber (61) is inserted into said first through passage (104), abutting against a shoulder (104a).

39. The method according to claim 33, during which the eukaryotic cells are hydrodynamically focused in the transportation channel so as to align them behind one another substantially in a hydrodynamic focusing plane (P) or substantially along a hydrodynamic focusing axis, and wherein the distance (D₁), between the outlet (102b) into the transportation channel (100) of the through passage (102) for the fluorescence excitation and said hydrodynamic focusing plane (P) or said hydrodynamic focusing axis of the eukaryotic cells (SP) in the transportation channel (100), is less than 1 mm, more preferably less than 100 μm, and still more preferably less than 50 μm.

40. The method according to claim 33, during which the eukaryotic cells are hydrodynamically focused in the transportation channel so as to align them behind one another substantially in a hydrodynamic focusing plane (P) or substantially along a hydrodynamic focusing axis, and wherein the distance between the outlet (103b) in the transportation channel (100) of the through passage (103) for the fluorescence detection and the hydrodynamic focusing plane (P) or the hydrodynamic focusing axis of the eukaryotic cells (SP) in the transportation channel (100), is less than 1 mm, more preferably less than 100 μm, and still more preferably less than 50 μm.
